(19) United States
(12) Patent Application Publication
(10) Pub. No.: US 2007/0135629 A1
(43) Pub. Date: Jun. 14, 2007

(54) Isolation of ansamitocins

(75) Inventor: Ashok D. Patil, Southborough, MA (US)

Correspondence Address: WOODCOCK WASHBURN LLP, CIRA CENTRE, 12TH FLOOR, 2929 ARCH STREET, PHILADELPHIA, PA, 19104-2891, US

(73) Assignee: Millennium Pharmaceuticals, Inc., Cambridge, MA (US)

(21) Appl. No.: 11/635,725

(22) Filed: Dec. 7, 2006

Related U.S. Application Data
(60) Provisional application No. 60/749,148, filed on Dec. 8, 2005.

(51) Int. Cl. C07D 491/14; C12P 17/00

(52) U.S. Cl. 540/462; 435/117

(57) ABSTRACT

The invention concerns a method having the steps of: combining an aqueous fermentation broth containing ansamitocins with a water-immiscible solvent and a water-soluble solvent to form an organic layer and an aqueous layer, wherein: (i) the water-immiscible solvent comprises at least 70% by volume of at least one solvent that is ethyl acetate, n-butylacetate, isopropylacetate, methyl ethyl ketone (MEK), methyl t-butyl ether, toluene, xylene or a mixture thereof,
(ii) the water-soluble solvent comprises at least one solvent that is C₁₋₅ alcohol, tetrahydrofuran, acetonitrile, or a mixture thereof, (iii) the ratio of the water-immiscible solvent to water-soluble solvent is in the range of 10:1 to 2:1 by volume, and (iv) the ratio of total solvent to fermentation broth is at least 2:1 by volume; partitioning the ansamitocins into the organic layer; separating the organic layer from the aqueous layer; and removing at least a portion of the solvent from the organic layer to provide an organic extract that comprises ansamitocins. The extract can be further manipulated to produce purified ansamitocins.

### CROSS-REFERENCE TO RELATED APPLICATIONS

This application claims benefit of U.S. Provisional Application No. 60/749,148, filed Dec. 8, 2005, the disclosure of which in incorporated herein by reference.

### FIELD OF THE INVENTION

The invention concerns methods for obtaining ansamitocins from a fermentation broth and compositions related thereto.

### BACKGROUND OF THE INVENTION

A mixture of structurally related ansamitocins have been produced from cultures utilizing Actinosynnema spp. such as Actinosynnema pretiosum. These processes are described, for example, in U.S. Pat. Nos. 4,450,234, 4,356,265, 4,228,239, and 4,162,940. Ansamitocins known to be produced by these processes are represented by the following formula: 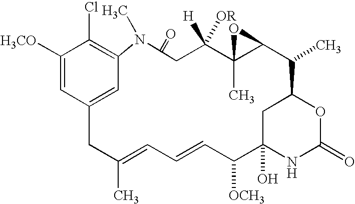
where R is a short aliphatic acyl group. These compounds include those where R is isobutyryl (designated as P-3 or AP-3), ethionyl (P-1), propionyl (P-2), butyryl (P-3′), isovaleryl (P-4), and valeryl (P-4′). See, for example, U.S. Pat. No. 6,573,074. Reductive cleavage of any of these acyl groups provides maytansinol, where R is hydrogen. Maytansinol is useful in the treatment of cancer. See, for example, U.S. Patent Application No. 20050113571.

Prior art methods for obtaining these ansamitocin precursors to maytansinol are low-yielding and costly. The ansamitocins must be purified from a complex fermentation broth. The purification methods typically require tedious chromatography steps and the overall process is time-consuming. There is a need for improved methods of isolating ansamitocins from their fermentation broth, especially methods that provide better yields. In particular, there is a need for improved methods that avoid messy or intractable emulsions, as well as, avoid tedious chromatography.

### SUMMARY OF THE INVENTION

This invention provides an improved method of extracting ansamitocins from a fermentation broth. The method avoids intractable emulsions and tedious chromatography. The ansamitocins are concentrated in an organic extract, which is then useful for the further purification of the ansamitocin. In one aspect, the invention concerns a method comprising:
combining an aqueous fermentation broth containing ansamitocins with a water-immiscible solvent and a water-soluble solvent to form an organic layer and an aqueous layer, wherein:
   (i) the water-immiscible solvent comprises at least 70% by volume of at least one solvent that is ethyl acetate, n-butylacetate, isopropylacetate, methyl ethyl ketone (MEK), methyl t-butyl ether, toluene, xylene or a mixture thereof,
   (ii) the water-soluble solvent comprises at least one solvent that is C₁₋₅ alcohol, tetrahydrofuran (THF), acetonitrile, or a mixture thereof,
   (iii) the ratio of the water-immiscible solvent to water-soluble solvent is in the range of from 10:1 to 2:1 by volume, and
   (iv) the ratio of total solvent to fermentation broth is at least 2:1 by volume;
partitioning the ansamitocins into the organic layer;
separating the organic layer from the aqueous layer; and
removing at least a portion of the solvent from the organic layer to provide an organic extract that comprises ansamitocins.

In some embodiments, the ansamitocins comprise at least one compound of formula I: 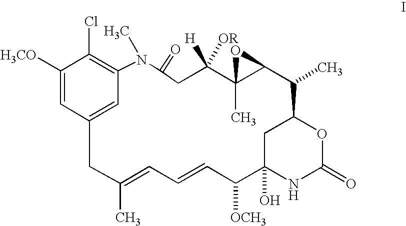
where R is —CO-ethyl, —CO-propyl, —CO-isopropyl, or —CO-butyl.

The partitioning of the ansamitocins into the organic layer can be an active step such as agitating or stirring the organic and aqueous layers. It is understood that some partitioning of the ansamitocins into the organic layer is likely to be accomplished in the absence of a separate, active step as a result of the contact between the organic and aqueous layers that form when the solvents are combined with the fermentation broth. The active step such as stirring or agitating is preferred to help facilitate the partitioning.

In certain embodiments, the water-immiscible solvent comprises ethyl acetate. In some embodiments, the water-soluble solvent comprises methanol.

Some methods utilize a ratio of water-immiscible solvent to water-soluble solvent that is about 8:1 to about 4:1 by volume. In yet other methods, the ratio of water-immiscible solvent to water-soluble solvent is about 6:1 to about 4:1 by volume. Certain methods use a ratio of total solvent to fermentation broth of at least 3:1 by volume.

In some embodiments, the method further comprises the steps:
combining the organic extract with a second water-immiscible solvent and optionally water to provide a second organic layer and a second aqueous layer, wherein the ansamitocins have a solubility in the second water-immiscible solvent of at least 10 mg/mL at 25° C.;
separating the second organic layer from the second aqueous layer; and
concentrating the second organic layer to produce a residue comprising ansamitocins.

In some embodiments, the ansamitocins have a solubility in the second water-immiscible solvent of at least 50 mg/mL at 25° C. The addition of water to the organic extract is optional because, depending on the method used to obtain the organic extract, sufficient water may be present to form the second aqueous phase without additional water being added.

In certain embodiments, the method further comprises the steps:
dissolving the residue in a third water-immiscible solvent that is ethyl acetate, isopropylacetate, n-butylacetate, toluene, xylene, or a mixture thereof to produce a third organic layer and a third aqueous layer;
adding a C₅-C₁₀ hydrocarbon solvent to the third organic layer to produce a precipitate containing ansamitocins; and
isolating the precipitate;
the ratio of C₅-C₁₀ hydrocarbon to third water-immiscible solvent being about 10:1 to about 3:1 by volume and the amount of third water-immiscible solvent being at least 5 mL per gram of the residue.

The C₅-C₁₀ hydrocarbon solvent can be, for example, pentane, hexane, heptane, cyclohexane, or mixtures thereof. In some embodiments, the C₅-C₁₀ hydrocarbon solvent is heptane.

In certain embodiments, the third water-immiscible solvent is ethyl acetate.

The ratio of C₅-C₁₀ hydrocarbon solvent to third water-immiscible solvent is about 6:1 to about 3:1 by volume in some embodiments. At least 10 mL of the third water-immiscible solvent per gram of residue is used in certain embodiments of the invention.

The invention can further comprise the steps:
contacting the precipitate with dichloromethane (DCM) or acetonitrile to provide (a) a solution comprising ansamitocins and (b) insoluble materials;
separating the solution from the insoluble materials; and
isolating at least a portion of the ansamitocins from the solution.

In another aspect, the invention concerns a composition comprising:
20-60% by weight of ansamitocins;
40-60% by weight of C₁₂-C₂₀ saturated and unsaturated fatty acids; and
1-10% by weight of phthalates of C₁₂-C₂₀ fatty acids.

In one embodiment, the ansamitocins comprise at least one compound of formula I above.

The invention also relates to a composition comprising:
30-70% by weight of a first compound of formula II: 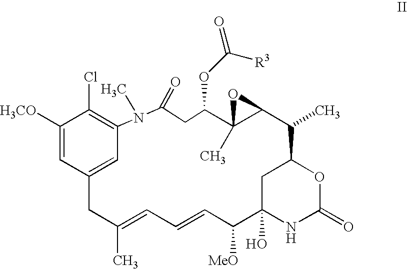
   where each R³ is independently C₁-C₄ alkyl; and
2-10% by weight of a second compound of formula III: 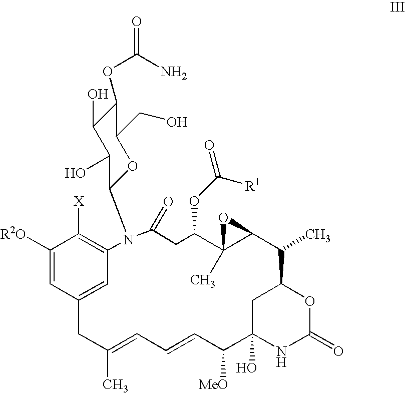
   wherein each R¹ is C₁-C₄ alkyl; R² is H or methyl; and X is H or Cl. The remaining components of the composition may include sugars and polysaccharides. In some embodiments, these remaining components make up about 20 to about 50% by weight of the composition.

In yet another aspect, the invention concerns a compound of formula IV: 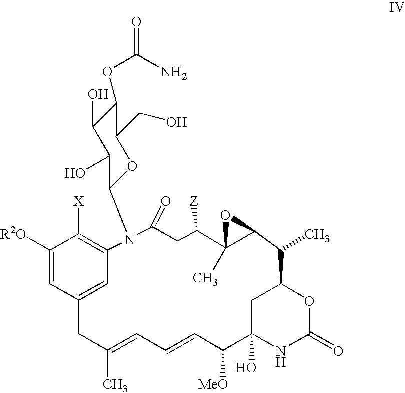
where Z is O(C═O)R¹; each R¹ is independently C₁-C₄ alkyl; R² is H or methyl; and X is H or Cl.

### BRIEF DESCRIPTION OF THE DRAWINGS

FIG. 1 shows an embodiment of the overall process for obtaining purified ansamitocins.

### DETAILED DESCRIPTION OF ILLUSTRATIVE EMBODIMENTS

This invention concerns the purification of ansamitocins. Ansamitocins are produced by methods well known to those skilled in the art. See, for example, U.S. Pat. Nos. 4,450,234, 4,356,265, 4,228,239, and 4,162,940. This invention is useful for purifying ansamitocins that include those compounds of the formula I: 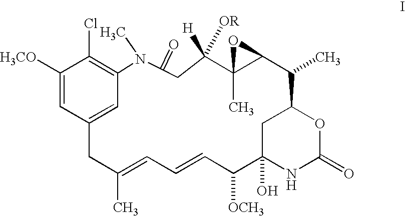
where R is —CO-ethyl, —CO-propyl, —CO-isopropyl, or —CO-isobutyl.

The term “alkyl”, used alone or as part of a larger moiety, includes both linear and branched chains. Some preferred alkyl groups have I to 4 carbon atoms. In some compositions, the alkyl group has 2 to 4 carbon atoms. Examples of alkyl groups include, but are not limited to, methyl, ethyl, n-propyl, isopropyl, n-butyl, isobutyl, sec-butyl, and t-butyl.

Particular compounds of formula I may also be designated as follows: AP2, where R is CO—CH₂CH₃; AP3, where R is CO—CH(CH₃)₂; AP3′, where R is CO—CH₂CH₂CH₃; and AP4, where R is CO—CH₂CH(CH₃)₂.

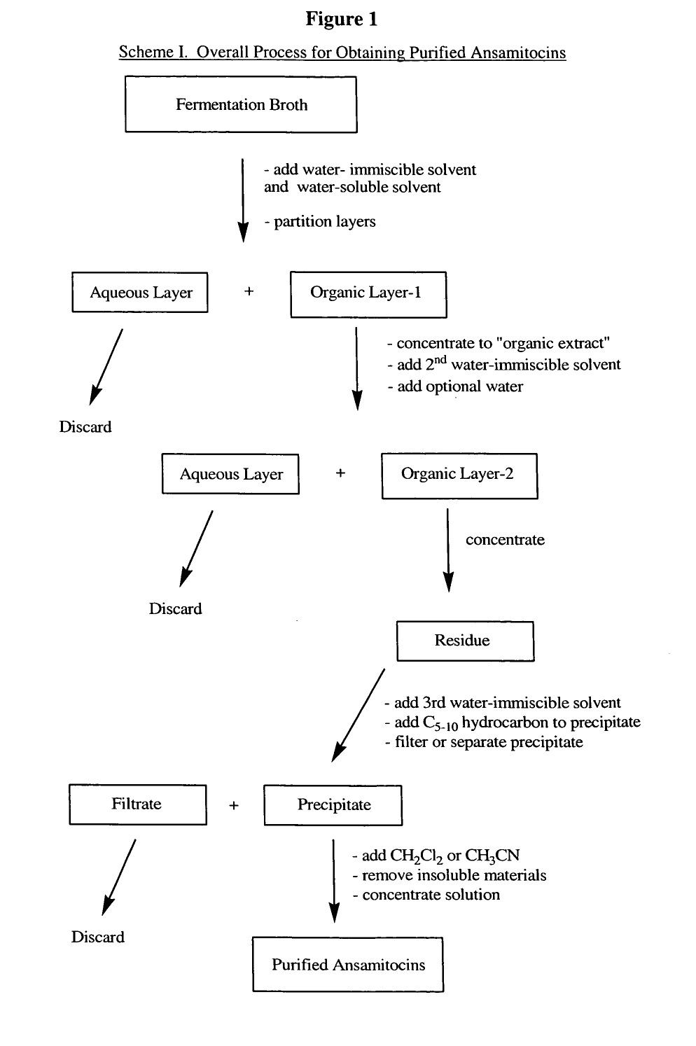

A general overall process for obtaining purified ansamitocins of formula I starting with bacterial fermentation broth is shown in Scheme I/FIG. 1. It has now been found that certain water-immiscible solvents and water-soluble solvents in certain quantities can be used effectively to partition the fermentation broth into separable organic and aqueous layers whereby the desired ansamitocins reside in the organic layer. After separation of the layers, the organic layer is concentrated and further partitioned between water and a second water-immiscible solvent to provide a second organic layer, which is then separated and concentrated to give a crude residue containing the ansamitocins. The residue may then be re-dissolved in a third water-immiscible solvent to provide a solution of ansamitocins along with unwanted impurities. Upon the addition of a C₅₋₁₀ hydrocarbon solvent, the impurities will precipitate from the solution and can be conveniently removed to leave behind a solution of pure ansamitocins. Compared to prior art methods, the present process can be performed more quickly and with better yields of the ansamitocins. It is notable that pure ansamitocins can be obtained without the need for chromatography.

According to this process, ansamitocins can be isolated by a method having the steps:
combining an aqueous fermentation broth containing ansamitocins with a water-immiscible solvent and a water-soluble solvent to form an organic layer and an aqueous layer, wherein:
   (i) the water-immiscible solvent comprises at least 70% by volume of at least one solvent that is ethyl acetate, n-butylacetate, isopropylacetate, methyl ethyl ketone (MEK), methyl t-butyl ether, toluene, xylene or a mixture thereof,
   (ii) the water-soluble solvent comprises at least one solvent that is C₁₋₅ alcohol (preferably, C₁₋₃ alcohol), tetrahydrofuran (THF), acetonitrile, or a mixture thereof,
   (iii) the ratio of the water-immiscible solvent to water-soluble solvent is in the range of from 10:1 to 2:1 by volume, and
   (iv) the ratio of total solvent to fermentation broth is at least 2:1 by volume;
partitioning the ansamitocins into the organic layer;
separating the organic layer from the aqueous layer; and
removing at least a portion of the solvent from the organic layer to provide an organic extract that comprises ansamitocins.

The method can further comprise the steps:
combining the organic extract with a second water-immiscible solvent and optionally water to provide a second organic layer and a second aqueous layer, wherein the ansamitocins have a solubility in the second water-immiscible solvent of at least 10 mg/mL at 25° C.;
separating the second organic layer from the second aqueous layer; and
concentrating the second organic layer to produce a residue comprising ansamitocins.

The residue can comprise about 20 to about 60% by weight of ansamitocins; about 40 to about 60% by weight of C₁₂-C₂₀ saturated and unsaturated fatty acids; and about 1 to about 10% by weight of phthalates of C₁₂-C₂₀ fatty acids. The fatty acids are those that are typically found in bacterial fermentation broths, especially in those species known to be closely related to Actinosynnema, though the relative amounts will vary depending on the organism and fermentation conditions. The fatty acids may include hexadecanoic acid, palmitic acid, oleic acid, phthalic acid and alkyl esters thereof. Phthalates are typically in the form of dialkylphthalates, such as branched and straight chain di-(C₈-alkyl) phthalates.

In some embodiments, the ansamitocins have a solubility in the second water-immiscible solvent of at least 50 mg/mL at 25° C.

The addition of water to the organic extract is optional because, depending on the method used to obtain the organic extract, sufficient water may be present to form the second aqueous phase without additional water being added. For example, if the organic extract was obtained by using rotary evaporation of the organic layer, then water usually is present in sufficient amount to form the second aqueous phase and no additional water is needed. Alternatively, the organic extract may be obtained by concentrating the organic layer using a falling film evaporator (FFE) as described in U.S. Pat. No. 6,573,074 (see column 5, lines 37-44). When FFE or similar thin layer methods are used to obtain the organic extract, additional water typically is needed to form the second aqueous phase.

In some embodiments, the second water-immiscible solvent is ethyl acetate, n-butylacetate, isopropylacetate, methyl ethyl ketone (MEK), methyl t-butyl ether, toluene, xylene, dichloromethane, diethyl ether or a mixture thereof

In certain embodiments, the method further comprises the steps:
dissolving the residue in a third water-immiscible solvent that is ethyl acetate, isopropylacetate, n-butylacetate, toluene, xylene, or a mixture thereof to produce a third organic layer and a third aqueous layer;
adding a C₅-C₁₀ hydrocarbon solvent to the third organic layer to produce a precipitate containing ansamitocins; and
isolating the precipitate;
the ratio of C₅-C₁₀ hydrocarbon to third water-immiscible solvent being about 10:1 to about 3:1 by volume and the amount of third water-immiscible solvent being at least 5 mL per gram of the residue.

The invention can also be practiced with the following additional steps:
contacting the precipitate with dichloromethane or acetonitrile to provide (a) a solution comprising ansamitocins and (b) insoluble materials;
separating the solution from the insoluble materials; and
isolating at least a portion of the ansamitocins from the solution.

The invention is also directed to compounds of the formula: 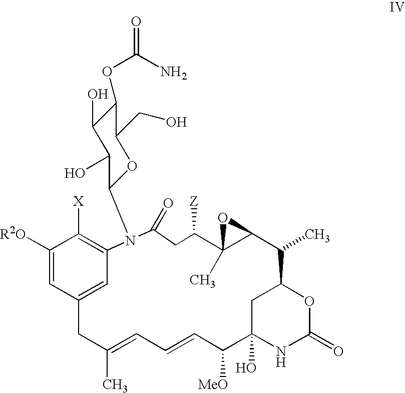
where Z is O(C═O)R¹; each R¹ is independently C₁-C₄ alkyl; R² is H or methyl; and X is H or Cl. These compounds are produced by the process of the invention.

Some compositions disclosed herein comprise:
30-70% by weight of a first compound of formula II: 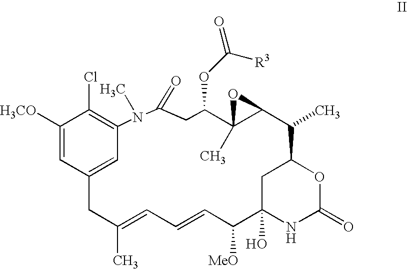
   where each R³ is independently C₁-C₄ alkyl; and
2-10% by weight of a second compound of formula III: 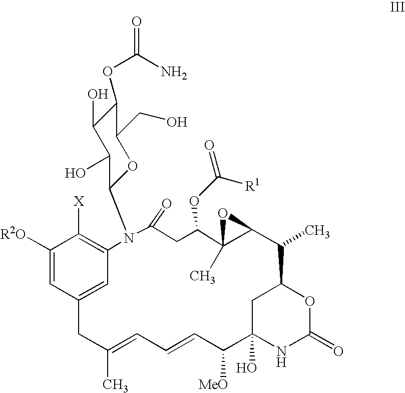
   where each R¹ is C₁-C₄ alkyl; R² is H or methyl; and X is H or Cl.

The first and second compounds may be isolated from the rest of the precipitate by column chromatography using a 25 cm C-18 reverse phase column and eluting with a water and acetonitrile gradient with 0.1% formic acid. The first compound elutes with 30:70 water to acetonitrile. The second compound elutes with 50:50 water to acetonitrile. The remaining components comprise 20-50% of the precipitate. These are various polar impurities such as sugars and polysaccharides. The remaining components are characterized as having no UV chromophore and may be eluted from the aforementioned column using 95:5 water to acetonitrile with 0.1% formic acid.

The acyl portion of the —O—C(═O)—R group can be reductively cleaved to form the corresponding alcohol. Reductive cleavage procedures are well known in the art. DIBAL (disobutylaluminum hydride) is an example of a suitable reducing agent. Such compounds produced by the reductive cleavage include those of formula V: 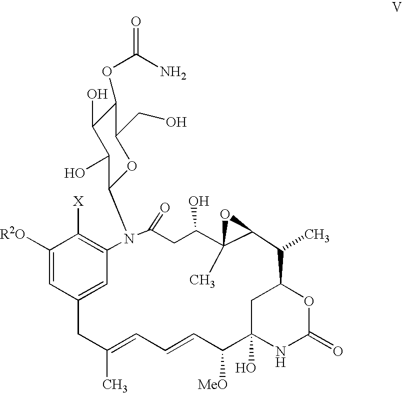
where R² is H or methyl; and X is H or Cl.

The invention is illustrated by the following examples that are not intended to limit the scope of the invention.

### EXAMPLES

The fermentation broth was obtained from IRL Biopharm Limited of Wellington, New Zealand.

### Example 1

Ethyl acetate (1250 mL) and methanol (250 mL) was placed in cylindrical jacketed vessel. Bacterial broth (500 mL) was gradually added to the solvent and the mixture agitated with glass stirrer. Initially stirring was gentle (RPM 110 for 5 minutes) and subsequently increased to 245 RPM for 2 hours. This combination of solvent to broth did not form emulsion and complete extraction of APs was efficiently completed in 2 hrs. A small amount (1 mL) of the organic layer was analyzed at 15, 30, 45 min, 1 and 2 hr. The dried solid was suspended in acetonitrile (1 mL), filtered through nylon-66 (0.44 micron) filter and analyzed by LC-MS using UV and ELSD (Evaporating Light Scattering Detector) for detection. At each of these points, the concentration of AP3 was slightly higher than 400 mg/L of the broth. In addition, it was noted that complete transfer of APs into the organic phase takes place in 15 minutes as concluded from time course study.

After 2 hours, stirring was suspended. Aqueous and organic layers were formed within minutes. The aqueous layer was discarded and the organic layer transferred to a round bottom flask (3 L) and solvent evaporated on a rotary evaporator at 40° C. After removal of ethyl acetate and methanol, most of the APs, oils, phthalates and other impurities became insoluble and were suspended in the remaining water (75 mL). At this stage, fresh ethyl acetate (100 mL) was added to the water to yield an aqueous and an organic layer. The ethyl acetate layer which appeared cloudy was filtered through a thin bed of celite and solvent evaporated to dryness to afford a brown residue (1.05 g). This residue was dissolved in hot ethyl acetate (10 mL) under stirring and precipitated with n-heptane (40 mL). The solution became cloudy and settled down in about two hours and then was filtered through a Whatman filter paper to give pale yellow solid and the filtrate. The filtrate was concentrated (0.53 g) and analyzed by LC-MS using ELSD which indicated that about 5% of AP3 was still left in the filtrate. No attempt was made to recover AP3 from the filtrate.

The pale yellow solid was suspended in dichloromethane (25 mL) and then filtered through Whatman filter paper. The filtrate was concentrated to yield residue (0.289 g). Analysis of this dichloromethane soluble portion by proton NMR and LC-MS suggested that this residue contained mainly AP3 (>95%) along with trace amounts of AP2, AP3′ and AP4. Analysis of the DCM insoluble portion indicated presence of polar compounds structurally related to AP3. Further purification of DCM insoluble residue using preparative reversed-phase HPLC and silica gel preparative TLC afforded four compounds of which one has been assigned novel structure as AP2-glucoside-4″-carbamate (1).

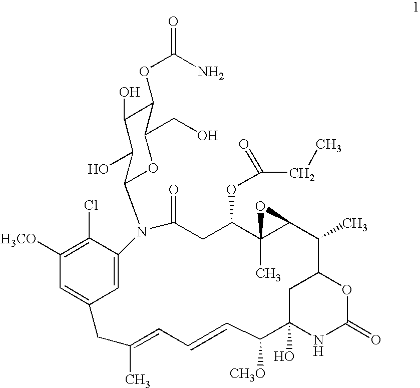

### Example 2

Ethyl acetate (3 L) and methanol (500 mL) were placed in a cylindrical jacketed vessel and the bacterial broth (1 L) was gradually added to the solvent. The mixture was agitated with a glass stirrer gently (RPM 115 for 5 minutes) initially and subsequently increased to 245 RPM for 2 hours. It was observed that this combination of solvent to broth did not form emulsion and complete extraction of APs took place efficiently within 2 hrs. It should be noted that in the event that an emulsion does form, additional methanol (200-500 mL) can be added.

After agitation was initiated, small amounts (1 mL) of the organic layer were removed from the mixture at the following intervals: 15 min, 30 min, 45 min, 1 hr and 2 hr. The samples were dried and then suspended in acetonitrile (1 mL). The suspension was filtered through nylon-66 (0.44 micron) and analyzed by LC-MS using UV and ELSD. At each of these points, the concentration of AP3 was found to be slightly higher than 400 mg/L of the broth. It was observed that complete transfer of APs into the organic phase took place in 15 minutes as observed from time course study.

After two hours, the stirring was suspended and the phases were allowed to separate and the aqueous phase was discarded. The organic layer was transferred to a round bottom flask (5 L) and the solvent was evaporated on a rotary evaporator at 40° C., until approximately 150 ml remained. The majority of the 150 ml was water. After removal of ethyl acetate and methanol, most of the APs, oils, phthalates and other impurities became insoluble and suspended in the remaining water (150 mL) forming an organic extract.

At this stage, fresh ethyl acetate (200 mL) was added to the remaining material from the previous step. The material was stirred, the phases were allowed to separate, and then the aqueous phase was discarded. The resulting cloudy ethyl acetate layer was then filtered through a thin bed of celite The solvent was evaporated to dryness to afford a brown residue (2.11 g).

The residue was dissolved in hot (55-60° C.) ethyl acetate (20 mL) with stirring. n-Heptane (80ml) was added to the ethyl acetate to form a precipitate. The solution became cloudy and settled down in about two hours. The solution was filtered through a Whatman filter paper to obtain pale yellow solid and the filtrate. The filtrate (1.06 g) was concentrated and analyzed by liquid chromatography-mass spectroscopy (LC-MS) using UV and Evaporating Light Scattering Detectors (ELSD). Analysis indicated that about 5% of AP3 was still left in the filtrate.

The yellow solid was dissolved in dichloromethane (50 mL) and then filtered through a Whatman filter paper. The dichlormethane insolubles were discarded (0.423 grams). The filtrate was concentrated to afford pure APs (0.567 g).

Analysis of this dichloromethane soluble portion by proton NMR and LC-MS suggested that this residue contained mainly AP3 (>95%) along with trace amounts of AP2, AP3′ and AP4. Analysis of the dichloromethane insoluble portion (0.423 g) indicated the presence of polar compounds some of them structurally related to AP3.

What is claimed:

1. A method comprising:
combining an aqueous fermentation broth containing ansamitocins with a water-immiscible solvent and a water-soluble solvent to form an organic layer and an aqueous layer, wherein:
(i) the water-immiscible solvent comprises at least 70% by volume of at least one solvent that is ethyl acetate, n-butylacetate, isopropylacetate, methyl ethyl ketone (MEK), methyl t-butyl ether, toluene, xylene or a mixture thereof,
(ii) the water-soluble solvent comprises at least one solvent that is C₁₋₅ alcohol, tetrahydrofuran, acetonitrile, or a mixture thereof,
(iii) the ratio of the water-immiscible solvent to water-soluble solvent is in the range of 10:1 to 2:1 by volume, and
(iv) the ratio of total solvent to fermentation broth is at least 2:1 by volume;
partitioning the ansamitocins into the organic layer;
separating the organic layer from the aqueous layer; and
removing at least a portion of the solvent from the organic layer to provide an organic extract that comprises ansamitocins.

2. The method of claim 1 wherein the ansamitocins comprise at least one compound of formula I: 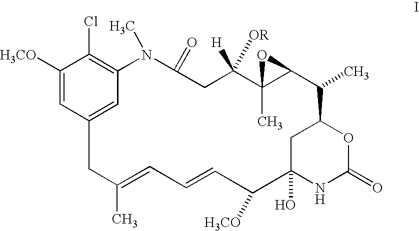
where R is —CO-ethyl, —CO-propyl, —CO-isopropyl, or —CO-isobutyl.

3. The method of claim 1 wherein the partitioning includes agitating or stirring the organic and aqueous layers.

4. The method of claim 1 wherein the water-immiscible solvent comprises ethyl acetate.

5. The method of claim 1 wherein the water-soluble solvent comprises methanol.

6. The method of claim 1 wherein the water-immiscible solvent comprises ethyl acetate and the water-soluble solvent comprises methanol.

7. The method of claim 1 wherein the ratio of water-immiscible solvent to water-soluble solvent is about 8:1 to about 4:1 by volume.

8. The method of claim 1 wherein the ratio of water-immiscible solvent to water-soluble solvent is about 6:1 to about 4:1 by volume.

9. The method of claim 1 wherein the ratio of total solvent to fermentation broth is at least 3:1 by volume.

10. The method of claim 1 further comprising the steps:
combining the organic extract with a second water-immiscible solvent and optionally water to provide a second organic layer and a second aqueous layer, wherein the ansamitocins have a solubility in the second water-immiscible solvent of at least 10 mg/mL at 25° C.;
separating the second organic layer from the second aqueous layer; and
concentrating the second organic layer to produce a residue comprising ansamitocins.

11. The method of claim 10 further comprising the steps:
dissolving the residue in a third water-immiscible solvent that is ethyl acetate, isopropylacetate, n-butylacetate, toluene, xylene, or a mixture thereof to produce a third organic layer and a third aqueous layer;
adding a C₅-C₁₀ hydrocarbon solvent to the third organic layer to produce a precipitate containing ansamitocins; and
isolating the precipitate;
the ratio of C₅-C₁₀ hydrocarbon to third water-immiscible solvent being about 10:1 to about 3:1 by volume and the amount of third water-immiscible solvent being at least 5 mL per gram of the residue.

12. The method of claim 11 wherein the C₅-C₁₀ hydrocarbon solvent is pentane, hexane, heptane, cyclohexane, or mixtures thereof.

13. The method of claim 12 wherein the C₅-C₁₀ hydrocarbon solvent is heptane.

14. The method of claim 11 wherein the third water-immiscible solvent is ethyl acetate.

15. The method of claim 11 wherein the C₅-C₁₀ hydrocarbon solvent is heptane and third water-immiscible solvent is ethyl acetate.

16. The method of claim 11 wherein the ratio of C₅-C₁₀ hydrocarbon solvent to third water-immiscible solvent is about 6:1 to about 3:1 by volume.

17. The method of claim 11 wherein at least 10 mL of the third water-immiscible solvent per gram of residue is used.

18. The method of claim 11 further comprising:
contacting the precipitate with dichloromethane or acetonitrile to provide:
a solution comprising ansamitocins, and
insoluble materials;
separating the solution from the insoluble materials; and
isolating at least a portion of the ansamitocins from the solution.

19. A composition comprising:
20-60% by weight of ansamitocins;
40-60% by weight of C₁₂-C₂₀ saturated and unsaturated fatty acids; and
1-10% by weight of phthalates of C₁₂-C₂₀ fatty acids.

20. The composition of claim 19 wherein the ansamitocins comprise at least one of compound of formula I: 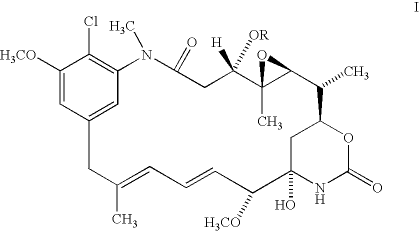
where R is-CO-ethyl, —CO-propyl, —CO-isopropyl, or —CO-isobutyl.

21. A composition comprising:
30-70% by weight of a first compound of formula II: 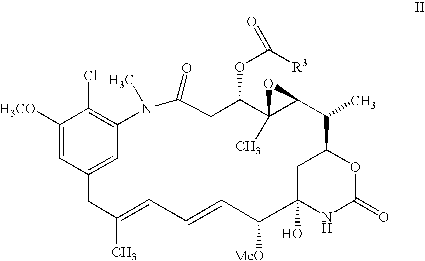
where each R³ is independently C₁-C₄ alkyl; and
2-10% by weight of a second compound of formula III: 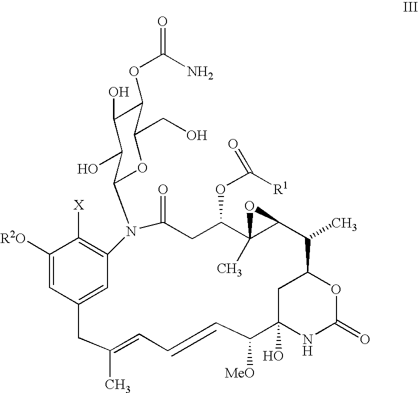
where each R¹ is C₁-C₄ alkyl; R² is H or methyl; and X is H or Cl;

22. A composition comprising a compound of formula IV: 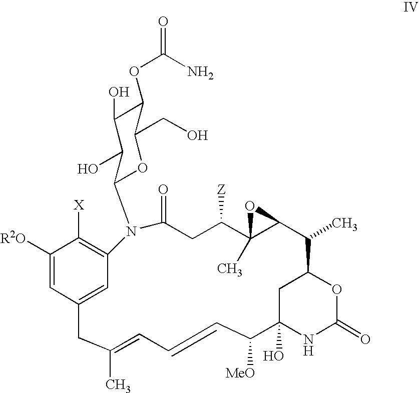
where Z is O(C═O)R¹ or OH; each R¹ is independently C₁-C₄ alkyl; R² is H or methyl; and X is H or Cl.
